(19) United States
(12) Patent Application Publication
(10) Pub. No.: US 2017/0303808 A1
(43) Pub. Date: Oct. 26, 2017

(54) Capacitive Electrocardiography (ECG) Systems

(71) Applicant: Medical Design Solutions, Inc., Milpitas, CA (US)

(72) Inventor: Robert T Stone, Sunnyvale, CA (US)

(21) Appl. No.: 15/491,808

(22) Filed: Apr. 19, 2017

Related U.S. Application Data
(60) Provisional application No. 62/325,488, filed on Apr. 21, 2016.

(51) Int. Cl. A61B 5/0408; A61B 5/01; A61B 5/0205; A61B 5/1455; A61B 5/00; A61B 5/04; A61B 5/044; A61B 5/024

(52) U.S. Cl. CPC A61B 5/0408; A61B 5/044; A61B 5/01; A61B 5/0205; A61B 5/6804; A61B 5/14551; A61B 5/0006; A61B 5/04012; A61B 5/024; A61B 5/6831; A61B 2562/0223

(57) ABSTRACT

An electrocardiography (ECG) physiological monitoring system including an ECG sensor assembly further having at least a first capacitive electrode fabric layer configured to electrically couple to the subject's skin and detect ECG signals, a second reference electrode fabric layer that is configured to shield the first capacitive electrode fabric layer from electromagnetic interference, an electronics module that is in direct communication with the ECG sensor assembly and programmed to control the ECG sensor assembly, process ECG signals therefrom, and wirelessly transmit the processed ECG signals, and transmission conductors that are configured to provide a signal communication path between the electronics module and the ECG sensor assembly.

### FIELD OF THE INVENTION

The present invention relates to systems and methods for monitoring physiological characteristics of a subject. More particularly, the present invention relates to systems and methods for determining physiological characteristics as a function of electrocardiography (ECG) characteristics signals.

### BACKGROUND OF THE INVENTION

As is well known in the art, ECG signals provide an effective means for determining one or more physiological characteristics of a mammalian subject. By monitoring the ECG signals of a subject, any deviation in a seminal physiological characteristic, e.g. heart rate, can be detected and immediately transmitted to a physician for analysis and prompt preventative action(s). ECG is thus one of the most widely used means for monitoring the health status of a subject.

Conventional clinical ECG systems typically employ 12-15 silver-silver chloride (Ag—AgCl) electrodes, i.e. wet ECG electrodes, which are affixed to specific regions of the chest, arms, or hands and legs. Wet ECG electrodes provide a wet conducting medium for charge transfer between the electrodes and the body. Adhesive tape is also often applied to keep the wet ECG electrodes in place.

There are, however, several drawbacks and disadvantages associated with wet ECG electrodes and, hence, ECG systems employing same. A major drawback is that wet electrode ECG systems often require cleaning of the attachment site on the body and, in some instances, also shaving hair off the attachment site. Cleaning of the attachment site is inconvenient and can cause skin irritation, allergic reactions, and inflammation due to toxicological issues of the electrode gels in long-term treatments.

Additionally, the quality of the signal will be compromised as the electrode gel dehydrates during prolonged use.

Further, since it is difficult to keep the electrode gel and/or adhesives entirely separate from each other over the long term. Cross-coupling between neighboring electrode sites can thus occur through leakage current.

In an effort to overcome the drawbacks associated with wet electrode ECG systems, gel-less, i.e. dry, ECG electrode systems have been developed. Dry electrode ECG systems typically employ 4-12 capacitive electrodes and a reference electrode.

There are, however, similarly several drawbacks and disadvantages associated with dry ECG electrodes and, hence, ECG systems employing same.

A major drawback associated with dry ECG electrode systems is that the amplitude levels of ECG signals detected by a dry electrode are typically very low. Thus, detecting spontaneous changes in ECG signals is very difficult.

Further, the high impedance exhibited by dry ECG electrodes necessitates an on-site amplifier with ultra-high impedance. Since the level of electrical noise introduced by the on-site amplifier is proportional to the magnitude of the impedances, signal quality of the amplifier output is degraded.

Dry ECG electrode systems also often exhibit poor signal quality due to external signal interference; particularly, electromagnetic radiation.

Dry electrode systems are also subject to internal signal interference that is generated by changes in the displacement of the electrode-to-skin distance and friction between the electrodes and the medium that the electrodes are attached to, e.g. fabric of a garment.

A further drawback associated with dry ECG electrode systems is that the systems typically employ a remote reference electrode that is positioned in such a manner that the body of a subject is disposed between the dry ECG electrode(s) and reference electrode.

By way of example, one conventional ECG system, known as the Electric Potential Integrated Circuit (EPIC) sensor system, which is produced by Plessey Semiconductors Ltd. (Application Note 291566, Issue No. 1), is designed and configured to be employed in a seat. The Plessey EPIC sensor system employs a large reference electrode that is incorporated into the seat and two ECG sensors that are incorporated into the clothing of an individual. To receive ECG signals with the Plessey EPIC sensor system, a subject must be positioned in the signal path between the reference electrode and the two ECG sensors.

Although the Plessey EPIC sensor system reduces external signal interference, internal signal interference is amplified.

Another major drawback of the EPIC sensor system is that the system only utilizes the “best” or “cleanest” ECG signal that is detected by one of the ECG sensors, while discarding the ECG signal that is detected by the other ECG electrode. As a result, the EPIC sensor system provides an ECG signal having substantially reduced accuracy due to the inherent subjectivity of the signal processing method.

It would thus be desirable to provide an improved ECG physiological monitoring system and method that (i) accurately monitors ECG signals representing one or more physiological characteristics associated with a user or wearer, (ii) does not require the use of any conductive gels, (iii) is suitable for long term monitoring of physiological characteristics, (iv) substantially reduces or abates external electromagnetic interference and (v) substantially reduces or abates internal interference.

It is therefore an object of the present invention to provide an improved ECG physiological monitoring system and method that (i) accurately monitors ECG signals representing one or more physiological characteristics associated with a user or wearer, (ii) does not require the use of any conductive gels, (iii) is suitable for long term monitoring of physiological characteristics, (iv) substantially reduces or abates external electromagnetic interference and (v) substantially reduces or abates internal interference.

It is another object of the present invention to provide an ECG physiological monitoring system and method that is suitable for long term monitoring of physiological characteristics.

It is another object of the present invention to provide an ECG physiological monitoring system and method that includes reliable and effective means to connect external modules, e.g. processing units.

### SUMMARY OF THE INVENTION

The present invention is directed to an improved system and method for determining physiological characteristics as a function of electrocardiography (ECG) characteristics signals.

In one embodiment of the invention, the ECG physiological monitoring system includes an ECG sensor system comprising a multilayer fabric having an outer fabric layer, a reference electrode fabric layer and an ECG electrode fabric layer.

Preferably, the reference electrode fabric layer provides an electromagnetic shield for the ECG electrode fabric layer. The reference electrode fabric layer preferably reduces external electromagnetic interference, such as interference due to the magnetic fields of power lines, by shielding the ECG electrode fabric layer.

In some embodiments, the ECG sensor system also comprises a single layer fabric comprising the reference electrode fabric layer.

In some embodiments, a predetermined a region of the reference electrode fabric layer is chemically processed to denature the conductance of the conductive fibers to form at least one chemically processed ECG electrode. In some embodiments, the conductive fibers are chemically processed with an oxidizing agent including, without limitation, chlorine and hydrogen peroxide.

In some embodiments, the ECG sensor system is in direct communication with an electronics module that preferably includes a processing system, which is programmed and configured to control the ECG sensor system and the function thereof, and the transmission and receipt of signals therefrom.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a top plane view of a section one embodiment of an ECG sensor system, in accordance with one embodiment of the invention;

FIG. 2 is a top plane view of one embodiment of a wearable ECG physiological monitoring system employing the ECG sensor system shown in FIG. 1, in accordance with the invention; and

FIG. 3 is a perspective view of another embodiment of a wearable ECG physiological monitoring system employing the ECG sensor system shown in FIG. 1, in accordance with the invention.

### DETAILED DESCRIPTION OF THE PREFERRED EMBODIMENT

Before describing the present invention in detail, it is to be understood that this invention is not limited to particularly exemplified apparatus, systems, structures or methods as such may, of course, vary. Thus, although a number of apparatus, systems and methods similar or equivalent to those described herein can be used in the practice of the present invention, the preferred apparatus, systems, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a sensor signal” includes two or more such signals and the like.

### Definitions

The term “monitor”, as used herein, means and includes, without limitation, one-time and continuous detection of ECG signal(s) and, hence, physiological characteristics and parameters of a subject associated therewith over a defined period of time.

The terms “physiological parameter” and “physiological characteristic”, as used herein, mean and include, without limitation, electrical activity of the heart, electrical activity of other muscles, electrical activity of the brain, pulse rate, blood pressure, blood oxygen saturation level, skin temperature, and core temperature.

The term “electrode”, as used herein, means and includes, without limitation, any electrical conductor used to provide physical communication between the metallic and non-metallic materials and/or compositions that comprise a circuit.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

Although the ECG systems and associated methods of the invention may be described in connection with monitoring ECG signals representing a defined physiological characteristic, e.g. heart rate, it is understood that the ECG systems and associated methods of the invention can be employed to monitor ECG signals representing various physiological characteristics.

It is further understood that although the ECG systems and associated methods of the invention are described herein in connection with a wearable garment, bed and car seat, the invention is not limited to such use/applications. The physiological monitoring systems and associated methods of the invention can also be employed in any other apparatus or system that maintains physical communication with a subject.

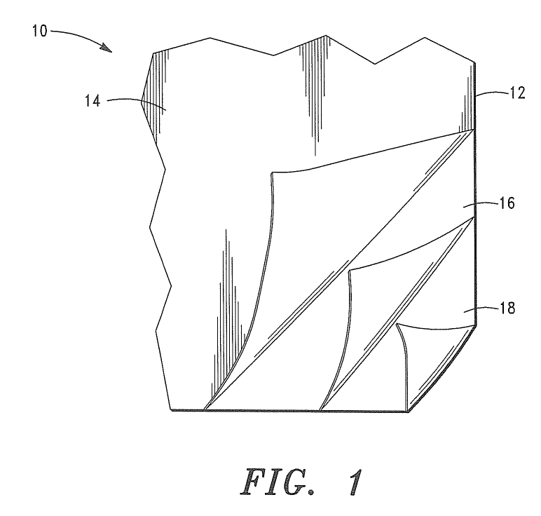

Referring now to FIG. 1, there is shown one embodiment of an ECG sensor system (or assembly) of the invention. As illustrated in FIG. 1, in a preferred embodiment, the ECG sensor system 10 comprises a multilayer fabric 12 having an outer fabric layer 14, a reference electrode fabric layer 16 and an ECG electrode fabric layer 18.

Preferably, the reference electrode fabric layer 16 provides an electromagnetic shield for the ECG electrode fabric layer 18. The reference electrode fabric layer 16 thus reduces external electromagnetic interference, such as interference due to the magnetic fields of power lines, by shielding the ECG electrode fabric layer 18. By positioning the reference electrode fabric layer 16 between the ECG electrode fabric layer 18 and the skin of a monitored subject, signal interference due to the displacement of the electrode-to-skin distance is also reduced.

According to the invention, the outer fabric layer 14 can comprise any conventional fabric, such as cotton or polyester.

According to the invention, the reference electrode fabric layer 16 can comprise a fabric selected from the group comprising, without limitation silver coated polyesters, carbon-impregnated polymers and conductive rubbers.

According to the invention, the ECG electrode fabric layer 18 can similarly comprise a fabric selected from the group comprising, without limitation silver coated polyesters, carbon-impregnated polymers and conductive rubbers.

According to the invention, the ECG sensor system 10 can also comprise a single layer fabric comprising the reference electrode fabric layer 16. In some embodiments, conductive fibers are woven into at least one predetermined region of the reference electrode fabric layer 16 to provide integrated ECG electrodes.

In some embodiments, a predetermined region of the reference electrode fabric layer 16 is chemically processed to denature the conductance of the conductive fibers to form at least one chemically processed ECG electrode. In some embodiments, the conductive fibers are chemically processed with an oxidizing agent including, without limitation, chlorine and hydrogen peroxide.

According to the invention, ECG sensor system 10 can be incorporated into a wearable ECG physiological monitoring system, such as, without limitation, the ECG physiological monitoring systems described in U.S. Application Nos. 62/325,290. filed on Apr. 20, 2016; which is expressly incorporated by reference herein in its entirety. The ECG sensor system 10 can also be incorporated into any apparatus or system comprising a fabric and/or any upholstered object.

According to the invention, ECG sensor system 10 can be employed in connection with the physiological monitoring system described in U.S. application Ser. No. 13/854,280, filed on Apr. 1, 2013; which is expressly incorporated by reference herein in its entirety.

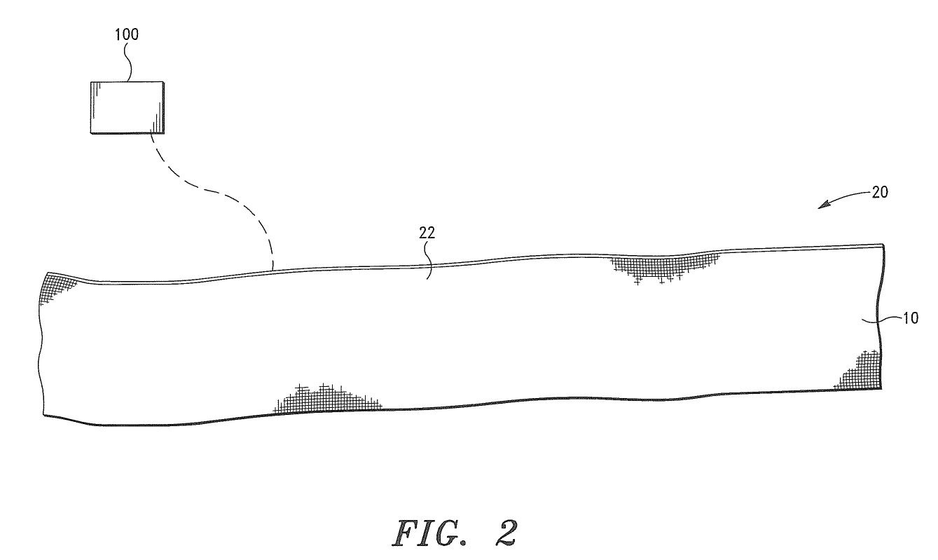

Referring now to FIG. 2, there is shown one embodiment of a wearable ECG physiological monitoring system 20 employing ECG system 10. As illustrated in FIG. 2, the system 20 comprises a band 22, which is configured to wrap around a subject's body, and an electronics module 100.

In some embodiments, wearable ECG physiological monitoring system 20 further includes one or more additional physiological sensors as described in U.S. Application Nos. 62/325,290 and Ser. No. 13/854,280, such as a magnetometer, pulse oximeter (SₚO₂) or core body temperature sensor.

According to the invention, the electronics module 100 can comprise an external or remote sub-system or be incorporated into the band 22.

As set forth in U.S. App. No. 62/325,290, the band 22 can comprise various conventional fabrics having fibers of variable loft and thickness.

As also set forth in U.S. App. No. 62/325,290, the electronics module 100 preferably includes at least one processing system, which is programmed and configured to control the ECG sensor system 10 and the function thereof, and the transmission and receipt of signals therefrom.

The processing system is also preferably programmed and configured to receive and process transmissions or signals from the ECG sensor system, and determine physiological information associated with a monitored subject (as a function of the signals), including at least one physiological characteristic, e.g. heart rate.

The electronics module 100 can further include a data transmission means that is programmed and configured to wirelessly transmit processed signals representing physiological characteristics to a remote signal receiving device (not shown), e.g., a base module or a hand-held electronic device, such as a smart phone, tablet, computer, wearable electronic, etc.

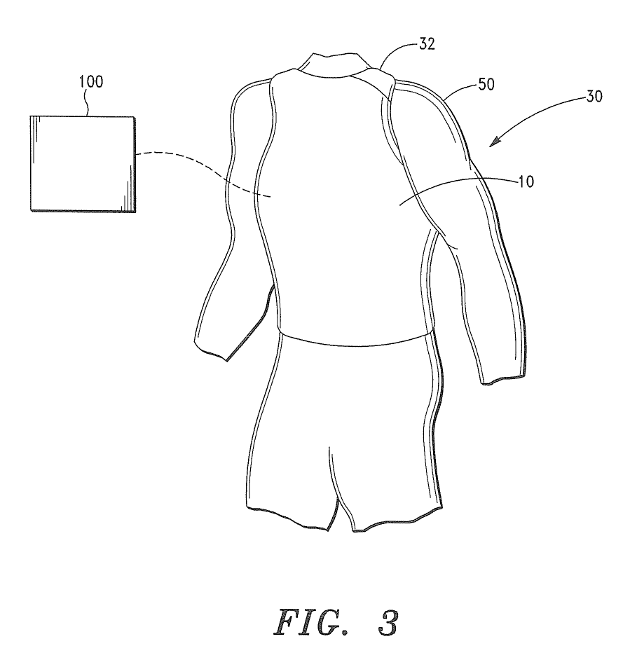

Referring now to FIG. 3, there is thus shown another embodiment of a wearable ECG physiological monitoring system 30. As illustrated in FIG. 3, the system 30 comprises a garment 32 comprising ECG sensor system 10 and the above discussed electronics module 100.

According to the invention, the electronics module can similarly comprise an external or remote sub-system or be incorporated into the garment 32.

As also illustrated in FIG. 3, in the noted embodiment, the garment 32 is preferably configured to cover at least the chest region and upper back of a subject 50. According to the invention, the garment 32 can, however, also be configured to cover other regions of the subject 50, including, without limitation, the lower abdominal region.

According to the invention, when the wearable ECG physiological monitoring systems 20, 30 are positioned on a subject, the systems 20, 30 accurately monitor ECG signals and characteristics associated with the subject, i.e. electrical activity associated with the subject, determine at least one physiological characteristic represented by the ECG signals, and transmit signals representing the physiological characteristic to a remote receiving and/or display device.

By virtue of the unique ECG “fabric” system and positioning of the monitored subject, the wearable ECG physiological monitoring systems described above provide several unexpected superior results and advantages:

The provision of wearable ECG physiological monitoring systems and methods that are suitable for long term, highly accurate monitoring of physiological characteristics.
The provision of wearable ECG physiological monitoring systems and methods that substantially reduce or abate external electromagnetic interference, e.g. electromagnetic radiation from any powered device, power lines and interstellar sources.
The provision of wearable ECG physiological monitoring systems and methods that substantially reduce or abate internal interference, e.g. interference due to changes in displacement of the electrode-to-skin distance and friction between the electrodes and the medium that the electrodes are attached to.
The provision of wearable ECG physiological monitoring systems and methods that provide a substantially greater (or enhanced) signal-to-noise (SNR) ratio compared to conventional ECG physiological monitoring systems.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An electrocardiography (ECG) physiological monitoring system, comprising:
an ECG sensor assembly that is configured to be positioned proximate a subject's skin;
said ECG sensor assembly comprising at least a first capacitive electrode fabric layer configured to electrically couple to said subject's skin and detect ECG signals,
said ECG sensor assembly comprising at least a second reference electrode fabric layer that is configured to shield said first capacitive electrode fabric layer from electromagnetic interference;
an electronics module comprising a processing system and a data transmission system, said electronic module being in direct communication with said ECG sensor assembly, said processing system including programs, instructions and associated algorithms and parameters to control said ECG sensor assembly, retrieve and process ECG signals transmitted by said ECG sensor assembly, determine physiological information associated with said subject as a function of said ECG signals, said data transmission system including a transmitter that is configured to wirelessly transmit said processed ECG signals; and
signal transmission conductors that are configured to provide a signal communication path between said electronics module and said ECG sensor assembly.

2. The ECG physiological monitoring system of claim 1, wherein said system includes a remote display device that is configured to receive and display said processed ECG signals from said electronics module.

3. The ECG physiological monitoring system of claim 1, wherein said system includes at least one additional physiological sensor that is in communication with said signal transmission conductors.

4. The ECG physiological monitoring system of claim 3, wherein said physiological sensor is selected from the group consisting of a magnetometer, pulse oximeter (SₚO₂) and core body temperature sensor.

5. An electrocardiography (ECG) physiological monitoring system, comprising:
an ECG sensor assembly that is configured to be positioned proximate a subject's skin;
said ECG sensor assembly comprising at least one reference electrode fabric layer;
said reference electrode fabric layer comprising first and second capacitive electrode fabric regions,
said first and second capacitive electrode fabric regions comprising conductive fibers woven therein that are configured to electrically couple to said subject's skin and detect ECG signals,
said reference electrode fabric layer being configured to average the capacitance potential of said first and second capacitive electrode fabric regions;
an electronics module comprising a processing system and a data transmission system, said electronics module being in direct communication with said ECG sensor assembly, said processing system including programs, instructions and associated algorithms and parameters to control said ECG sensor assembly, retrieve and process ECG signals transmitted by said ECG sensor assembly, determine physiological information associated with said subject as a function of said ECG signals, said data transmission system including a transmitter that is configured to wirelessly transmit said processed ECG signals; and
signal transmission conductors that are configured to provide a signal communication path between said electronics module and said ECG sensor assembly.

6. The ECG physiological monitoring system of claim 5, wherein said conductive fibers are chemically processed by an oxidizing agent selected from a group consisting of chlorine and hydrogen peroxide.

7. The ECG physiological monitoring system of claim 5, wherein said system includes a remote display device that is configured to receive and display said processed ECG signals from said electronics module.

8. The ECG physiological monitoring system of claim 5, wherein said system includes at least one additional physiological sensor that is in communication with said signal transmission conductors.

9. The ECG physiological monitoring system of claim 8, wherein said physiological sensor is selected from the group consisting of a magnetometer, pulse oximeter (SₚO₂) and core body temperature sensor.
